(19) United States
(12) Patent Application Publication
(10) Pub. No.: US 2026/0183753 A1
(43) Pub. Date: Jul. 2, 2026

(54) MIXED METAL OXIDE CATALYST SYSTEM FOR THE PARTIAL OXIDATION OF ALKANES TO VALUE ADDED PRODUCTS AND OXYGENATES

(71) Applicant: COUNCIL OF SCIENTIFIC AND INDUSTRIAL RESEARCH, New Delhi (IN)

(72) Inventors: Thirumalaiswamy RAJA, Pune (IN); Marimuthu PRABU, Pune (IN); Siva Prasad MEKALA, Pune (IN); Archana RAMAKRISHNAN, Pune (IN)

(73) Assignee: COUNCIL OF SCIENTIFIC AND INDUSTRIAL RESEARCH, New Delhi (IN)

(21) Appl. No.: 19/116,518

(22) PCT Filed: Sep. 29, 2023
(86) PCT No.: PCT/IN2023/050896

§ 371 Date: Mar. 28, 2025

(30) Foreign Application Priority Data
Sep. 29, 2022 (IN) 202211055893

(51) Int. Cl. B01J 23/75; B01J 21/04; B01J 23/00; B01J 23/745; B01J 23/86; B01J 23/889; B01J 35/61; B01J 35/64; B01J 37/00; B01J 37/02; B01J 37/03; B01J 37/04; B01J 37/08; C07C 29/48

(52) U.S. Cl. CPC B01J 23/75; B01J 21/04; B01J 23/005; B01J 23/745; B01J 23/862; B01J 23/8892; B01J 35/615; B01J 35/647; B01J 37/0036; B01J 37/009; B01J 37/0236; B01J 37/031; B01J 37/04; B01J 37/088; C07C 29/48

(57) ABSTRACT

The present invention relates to AB2O4 type mixed metal oxide catalyst system having spinel/inverse spinel structure with support. The present invention also relates to the process for preparation of mixed metal oxide catalyst system. More particularly, the present invention relates to a process for the partial oxidation of alkanes to value added products and corresponding oxygenates by using said AB2O4 type mixed metal oxide catalyst with support(s).

### FIELD OF THE INVENTION

The present invention relates to a AB₂O₄ type mixed metal oxide catalyst system having spinel/inverse spinel structure with support. Particularly, the present invention relates to a process for the partial oxidation of alkanes to value added products and corresponding oxygenates by using AB₂O₄ type mixed metal oxide catalyst with support(s).

### BACKGROUND AND PRIOR ART OF THE INVENTION

In nature, methane is converted into methanol in presence of enzymes at ambient conditions, unlike commercial processes. Methanol production is well-established process that commercially uses syn-gas as the precursor. Synthesis of methanol from syn-gas has major drawback that the process is economically viable only when produced on very large scale. Moreover, the production of syn-gas requires a very high temperature, thus making the entire process energy-intensive. Therefore, different approaches to methane evaluation have been explored in-depth, but commercialized routes preferred direct selective oxidation of methane. Different catalytic systems are employed for oxidation of methane which has been published with various oxidants for past few decades.

Reference may be made to Journal entitled “Aqueous Au—Pd colloids catalyze selective CH₄ oxidation to CH₃OH with O₂ under mild conditions” by Agarwal et al. and published in the journal “Science 358 (6360), 223-227” used colloidal gold-palladium nanoparticles supported on titanium oxide, and without support, the methanol selectivity (92%) in an aqueous solution at mild temperatures was obtained. Apparently, by using isotopically labelled gaseous phase oxygen as an oxidant in the presence of hydrogen peroxide, they found that the methanol incorporated around 70% substantial fraction of gas-phase O₂.

Reference may be made to Journal entitled “Hydrophobic zeolite modification for in situ peroxide formation in methane oxidation to methanol” by Jin et al. and published in the journal “Science 367, 193-197 (2020)” reported a heterogeneous catalyst for methanol production by methane oxidation by generating in situ hydrogen peroxide at low temperature (70° C.) with 17.3% conversion and 92% selectivity towards methanol. The productivity of methanol was about 91.6 mmoles per gram of AuPd catalyst per hour.

Reference may be made to Journal entitled “Dioxygen dissociation over man-made system at room temperature to form the active α-oxygen for methane oxidation” by Tabor et al. and published in the journal “Sci. Adv. 2020; 6: eaaz9776” reported cleavage of dioxygen at ambient reaction conditions using a binuclear Fe(II) species stabilized in an aluminosilicate matrix for methane activation. The formed α-oxygen species exhibited oxidation properties resulting in catalytic activity towards the methanol about 75 mmoles per gram of catalyst. This system is claimed to be a high potential for industrial use as the Fe(II)/Fe(IV) cycle is reversible, the active Fe centres are stable, and methanol can be released to the gas phase without any requirement of water or water-organic medium.

However, all the above-mentioned reports have certain drawbacks with respect to the scaling up of catalyst, cost economics of the catalyst, and reproducibility. Therefore, there is a need in the art to develop an industrially scalable, cheap process for alkane (methane) conversion by using cheap and easily available catalyst source.

### OBJECTIVES OF THE INVENTION

Main object of the present invention is to provide a mixed metal oxide catalyst system for partial oxidation of (C1-C4) alkanes into value added products having formula of AB₂O₄, having spinel/inverse spinel structure with support.

Another objective of the present invention is to provide a process for the partial oxidation of alkanes to value added products and corresponding oxygenates by using AB₂O₄ type mixed metal oxide catalyst with various supports.

### SUMMARY OF THE INVENTION

Accordingly, present invention provides a mixed metal oxide catalyst system for partial oxidation of (C₁-C₄) alkanes into value added products comprising:
i. spinel or inverse spinel structured mixed metal catalyst of formula AB₂O₄ supported onto a support;
   wherein
   A is a metal selected from the group consisting of manganese, cobalt, copper and chromium; B is a metal of iron;
   wherein the catalyst system exhibits array of ordered and crystallinity of cubic fd-3m symmetry.

In an embodiment of the present invention, the catalyst is in a phase having formula of AxBxO₄ supported onto support, wherein the support is in phase of 2-x, and wherein x is in range of 0.01 to 0.5.

In another embodiment of the present invention, the iron is in a ferrite form.

In yet another embodiment of the present invention, the support is selected from the group consisting of mesoporous alumina, MCM-41, ZSM-5, SBA-15 and mesoporous zirconium oxide.

In yet another embodiment of the present invention, the (C1-C4) alkane is selected from the group consisting of methane, ethane, propane and butane.

In yet another embodiment of the present invention, the value-added product is selected from (C1-C6) alcohol and aldehyde, either alone or mixture thereof.

In yet another embodiment of the present invention, the (C1-C6) alcohol is selected from the group consisting of methanol, ethanol, propanol, butanol, pentanol, and hexanol or mixtures thereof.

In yet another embodiment of the present invention, the aldehyde is selected from the group consisting of formaldehyde, acetaldehyde, propanal, butanal and pentanal or mixture thereof.

In yet another embodiment of the present invention, said catalyst system exhibit BET surface area in the range of 102.2 to 104.1 m²/g; average pore size in the range of 3.2 to 3.3 nm.

In yet another embodiment of the present invention, AB₂O₄ is dispersed onto surface of said support; Metal A has octahedral coordination sites, and metal B has tetrahedral and octahedral sites in said catalyst system; and/or Metal A is in +2 oxidation state, and metal B is in +3 oxidation state.

In yet another embodiment, present invention provides a process for the preparation of mixed metal oxide catalyst system, comprising the steps of:
a. dissolving the metal precursors in water in 1:1, 1:2, or 2:1 molar ratio under stirring for a time period in the range of 1.5 to 3 hours to obtain a solution;
b. adding 4 to 7M NaOH solution into the solution as obtained in step a) to obtain a reaction mixture and aging the reaction mixture for a time period in the range of 1.5 to 3 hours at a temperature in the range of 50 to 70° C.;
c. adding 2.5 to 4M NaOH solution into the reaction mixture obtained in step b) to obtain a product, followed by filtering and drying the product at a temperature in the range of 90 to 110° C. for a time period in the range of 20 to 26 hours and calcining at a temperature in the range of 520 to 580° C. in an air atmosphere for a time period in the range of 5 to 7 hours to obtained a catalyst without support;
d. mixing the catalyst obtained at step c) with support by using ball milling method for a time period in the range of 0.45 to 2 hours; and
e. calcining the catalyst with support obtained at step d) at a temperature in the range of 500 to 600° C. for a time period in the range of 5 to 7 hours to obtain the mixed metal oxide catalyst system.

In yet another embodiment of the present invention, Metal precursors at step a) is selected from the group consisting of iron nitrate, cobalt nitrate, Mn(NO₃)₂·4H₂O, Cu(NO₃)₂·3H₂O, and Cr (NO₃)₃·9H₂O.

In yet another embodiment, present invention provides a process for the partial oxidation of (C1-C4) alkane into value added products using the mixed metal oxide catalyst system of the present invention, the process comprising the steps of:
reacting the (C1-C4) alkane with an oxidant in a presence of water as solvent in a ratio ranging between 1:0.5:0.5 to 1:1:1.5 at pressure of oxidant in the range of 10-25 bar to obtain value added products.

In yet another embodiment of the present invention, the process is carried out in a batch mode in a Parr reactor(s) at a temperature in the range of 75-150° C. for a time period in the range of 5-7 hours or continuous mode in a continuous flow (FBR) reactor(s) at a temperature in the range of 150-500° C. for a time period in the range of 12-24 hours.

In yet another embodiment of the present invention, the oxidant is selected from the group consisting of CO₂, O₂ and H₂O₂.

In yet another embodiment of the present invention, the pressure of (C1-C4)alkane is in the range of 25-30 bar; and a gas hourly space velocity is in the range of 2000 to 4000 h⁻

In yet another embodiment the present invention, provides a process for the partial oxidation of alkanes to value added products and corresponding oxygenates.

In yet another embodiment the present invention, provides a process for the partial oxidation of methane to value added products in the presence of oxidants (O₂, CO₂, H₂O₂) by using AB₂O₄ type mixed metal oxide, spinel/inverse spinel structured catalyst with different supports, wherein A is selected from Mn, Co, Cu, Cr and B is Fe.

In yet another embodiment of the present invention, the process for the partial oxidation of methane by using AB₂O₄ type mixed metal oxide catalyst with support comprises of reacting methane with suitable oxidant in the presence of water at a temperature in the range of 75-150° C. for a time period in the range of 5-7 hours in a batch (Parr) and at a temperature in the range of 150-500° C. for a time period in the range of 12-24 hours for continuous flow (FBR) reactor.

### BRIEF DESCRIPTION OF THE DRAWINGS

FIG. 1 represents XRD of mesoporous Al₂O₃, CoFe₂O₄, 30% CoFe₂O₄ @ Al₂O₃ fresh and spent catalysts

FIG. 2 represents FESEM and EDAX pattern of (a) CoFe₂O₄ (b) CoFe₂O₄ on Al₂O₃ and (c) CoFe₂O₄ on Al₂O₃Spent

FIG. 3 represents a) The TEM Images and HAADF elemental mapping of CoFe₂O₄. b) HR-TEM images of (a and b) CoFe₂O₄@ Al₂O₃ and (c and d) CoFe₂O₄ @ Al₂O₃ spent

FIG. 4 represents NH₃ TPD of mesoporous Al₂O₃, CoFe₂O₄ and CoFe₂O₄ on Al₂O₃ fresh and spent

FIG. 5 represents XPS spectra of (a) CoFe₂O₄ (b) CoFe₂O₄ on Al₂O₃ (c) CoFe₂O₄ on Al₂O₃ spent catalysts

FIG. 6 represents a) FT IR spectra of CoFe₂O₄ on Al₂O₃ catalyst with CH₄ and O₂ with different temperature (50-350° C.). b) FT IR spectra of CoFe₂O₄ on Al₂O₃ catalyst with CH₄ and CO₂ with different temperature (50-350° C.).

FIG. 7 shows representative drawing of Parr reactor(s) used in the batch mode type of reaction for formation of oxygenated products using alkane and the catalyst system as disclosed herein.

FIG. 8 shows representative drawing of continuous flow (FBR) reactor(s) used in the continuous mode type of reaction for formation of oxygenated products using alkane and the catalyst system as disclosed herein.

### DETAILED DESCRIPTION OF THE INVENTION

The term “array of highly ordered” or “array of ordered” used herein refers to crystalline solids having regular ordered arrays of components (or in present case, metals) held together by uniform intermolecular forces. Whereas the components/metals in amorphous solids are not arranged in regular arrays.

The term “crystallinity of cubic fd-3m symmetry” used herein refers to crystalline nature of catalyst particles claimed in the specification having fd-3m symmetry where the fd-3m symmetry is well known in the art as space group 227 which follows the face-centered cubic Bravais lattice.

The present invention provides a process for the partial oxidation of alkanes to value added products and corresponding oxygenates by using AB₂O₄ type mixed metal oxide catalyst.

The present invention provides a mixed metal oxide catalyst system for partial oxidation of (C1-C4)alkanes into value added products, comprising:
spinel or inverse spinel structured mixed metal catalysts supported onto a support with formula represented as: AB₂O₄ onto support;
wherein the A and B are metals, wherein the metal B is iron, and
wherein the catalyst system exhibits array of ordered and crystallinity of cubic fd-3m symmetry.

The catalyst is in a phase having formula of AxBxMO₄ supported onto support, wherein the metal M of support is in phase of 2-x, and wherein x is in range of 0.01 to 0.5

The catalyst is in a phase having formula of CoxFexAl₂₋ₓO₄ supported onto support, wherein the support is in phase of Al₂₋ₓ, and wherein x is in range of 0.01 to 0.5

There is change in the oxidation state of Metal B i.e., Fe from Fe³⁺ to Fe²⁺, and metal A (e.g. cobalt from Co²⁺ to Co¹⁺) after supported onto said support due to said phase formation.

The mixed metal oxide catalyst system comprises one or more structural features, selected from:
a) BET surface area is in the range of 102.2 to 104.1 m²/g;
b) Average pore size is in the range of 3.2 to 3.3 nm;
c) B as iron is present in ferrite form;
d) AB₂O₄ is dispersed onto surface of said support;
e) Metal A has octahedral coordination sites, and metal B has tetrahedral and octahedral sites in said catalyst system; and/or
f) Metal A is in +2 oxidation state, and metal B is in +3 oxidation state in said catalyst system.

The metal A is selected from manganese, cobalt, copper and chromium.

The support is selected from mesoporous alumina, MCM-41, ZSM-5, SBA-15, and mesoporous zirconium oxide.

The present invention provides a process for the partial oxidation of (C1-C4) alkane into value added products using the mixed metal oxide catalyst system, the process comprises:
reacting the (C1-C4) alkane with suitable oxidant in a presence of water as solvent under reaction conditions to obtain value added products;
wherein the process is done in batch mode or continuous mode;
wherein in the batch mode, the reaction conditions kept at a temperature in the range of 75-150° C. for a time period in the range of 5-7 hours, or
in the continuous mode, the reaction conditions kept at a temperature in the range of 150-500° C. for a time period in the range of 12-24 hours.

The (C1-C4) alkane is selected from methane, ethane, propane and butane; and wherein the value added product is selected from (C1-C6) alcohol and aldehyde or mixture thereof.

The (C1-C6) alcohol is selected from methanol, ethanol, propanol, butanol, pentanol, hexanol or mixtures thereof, and the aldehyde is selected from formaldehyde, acetaldehyde, propanal, butanal, pentanal or mixture thereof.

The oxidant is selected from CO₂, O₂, and H₂O₂.

The ratio of (C1-C4) alkane:oxidant:water is in the range of 1:0.5:1 to 1:1:1.

The reaction conditions of the process for the partial oxidation of (C1-C4) alkane into value added products, further comprise one or more of:
i) a ratio of mixture of (C1-C4) alkane, oxidant and water is 1:0.5:Y, wherein Y is in the range of 0.5 to 1.5;
ii) gas hourly space velocity of mixture of (C1-C4) alkane, oxidant and water is in the range of 2000 to 4000 h⁻¹;
iii) pressure of (C1-C4) alkane is in the range of 25-30 bar; and
iv) pressure of oxidant is in the range of 10-25 bar;

The batch mode is done in a Parr reactor(s).

The continuous mode is done in a continuous flow (FBR) reactor(s).

The value added products are selected from alcohol or aldehyde, depends on type of alkane used.

The value added products are selected from methanol, ethanol and formaldehyde.

The present invention provides a process for the partial oxidation of methane to value added products in the presence of oxidants by using AB₂O₄ type mixed metal oxide, spinel/inverse spinel structured catalyst with different supports, wherein A is selected from Mn, Co, Cu, Cr and B is Fe.

The process for the partial oxidation of methane by using AB₂O₄ type mixed metal oxide catalyst with support comprises of reacting methane with suitable oxidant in the presence of water at a temperature in the range of 75-150° C. for a time period in the range of 5-7 hours in a batch (Parr) and at a temperature in the range of 150-500° C. for a time period in the range of 12-24 hours for continuous flow (FBR) reactor.

Oxidant is selected from CO₂, O₂, and H₂O₂.

Another aspect of the present invention is to provide a process for the preparation of a AB₂O₄ type mixed metal oxide catalyst with support, wherein the process comprises the steps of:
a) dissolving the metal precursors A and B in ratio of 1:1, 1:2, or 2:1 molar ratio in water under stirring for 2 hours;
b) adding 6M NaOH solution into the solution obtained at step a) and aging the reaction mixture for 2 hours at 60° C.;
c) adding 3M NaOH solution into the reaction mixture obtained at step b);
d) filtering and drying the product obtained at 100° C. for 24 hours and calcining at 550° C. in an air atmosphere for 6 hours to obtained the catalyst without support;
e) mixing the catalyst obtained at step d) with support by using ball milling method for 1 hour;
f) calcining the catalyst with support obtained at step f) at 550° C. for 6 hours to get the value added product.

Metal precursors at step a) is selected from iron nitrate, cobalt nitrate, Mn(NO₃)₂·4H₂O, Cu(NO₃)₂·3H₂O, or Cr (NO₃)₃·9H₂O. In preferred embodiment, metal precursor A is selected from cobalt nitrate, Mn(NO₃)₂·4H₂O, Cu(NO₃)₂·3H₂O, and Cr (NO₃)₃·9H₂O, and the metal precursor B is iron nitrate. In particularly useful embodiment, iron and cobalt nitrates metal precursors are used.

Support used at step e) is selected from mesoporous alumina, MCM-41, ZSM-5, SBA-15, and mesoporous zirconium oxide. In particularly useful embodiment, mesoporous alumina is used as a support.

Table 1 shows the BET surface area, pore sizes, and pore volume of freshly synthesized mesoporous alumina support and with fresh and spent ferrite catalysts. The mesoporous alumina support has a low surface area (80.1 m²/g) compared to CoFe₂O₄ supported on alumina (104.1 m²/g), which is due to the substitution of Co²⁺ and Fe³⁺ sites by Al³⁺ with a similar ionic radius (ionic radii=0.390 nm). Increasing in the pore volume is due to the isomorphic substitution of Fe³⁺, Co²⁺ by Al³⁺ which is clear evidence that the formation of new phase of CoxFexAl₂₋ₓO₄ where x=0.01 to 0.5.

TABLE 1

| | | | Average | Average |
|---|---|---|---|---|
| | | Surface area | pore | pore |
| S. No | Catalyst | (m²/g) | size (nm) | volume (cc/g) |
| 1. | Mesoporous Al₂O₃ | 80.3 | 3.3 | 0.11 |
| 2. | CoFe₂O₄ on Al₂O₃ | 104.1 | 3.3 | 0.15 |
| 3. | CoFe₂O₄ on Al₂O₃ | 102.2 | 3.2 | 0.16 |
| | spent | | | |

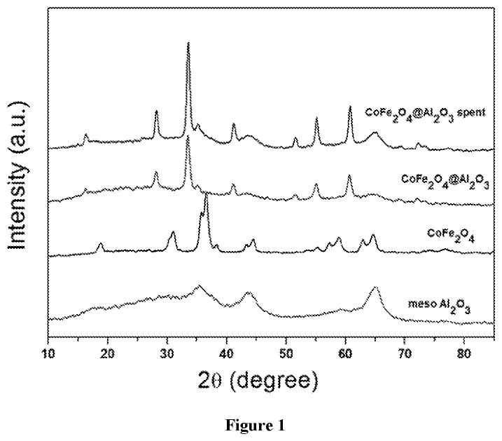

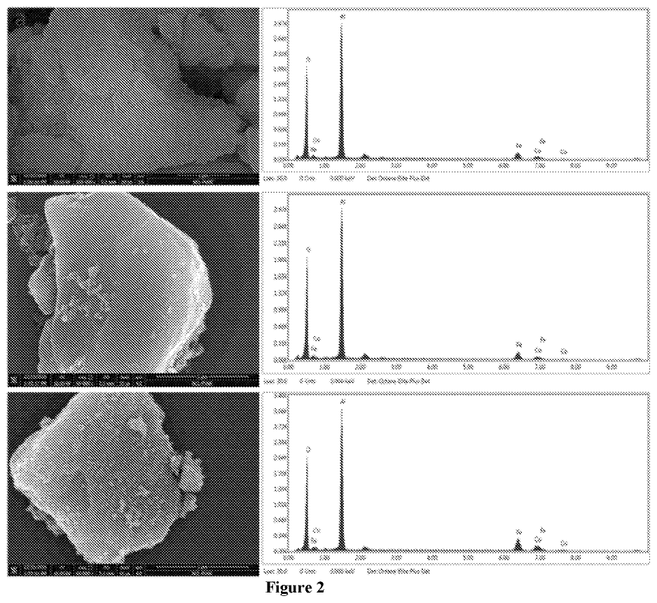

The FIG. 1 shows the X-ray diffractograms of mesoporous Al₂O₃, CoFe₂O₄, CoFe₂O₄ on Al₂O₃, and CoFe₂O₄ on Al₂O₃ spent. Mesoporous alumina shows the characteristic peaks corresponding to the formation of γ-Al₂O₃ at (311), (400), (511), and (440) as per JCPDS No: 10-0425. It indicates that pure γ-Al₂O₃ support is formed without any impurities. The characteristic peaks of CoFe₂O₄ inverse spinel correspond to (111), (220), (311), (400), (422), (511), and (440) are in well agreement with the JCPDS file (PDF 22-1086). After loading the ferrite on a mesoporous alumina support, a peak shift in the lower 2 θ angle due to the formation of new phases of CoₓFeₓAl₂₋ₓO₄ (X=0.01-0.5) is observed.

FESEM images of as-prepared CoFe₂O₄, CoFe₂O₄ on Al₂O₃, and CoFe₂O₄ on Al₂O₃ spent catalyst are shown in FIG. 2. SEM images of b & c show that fresh and spent catalysts supported on alumina have no changes in catalyst structure. The EDAX pattern gives clear evidence of Co, Fe, Al, and O are present without any metal oxides as impurities.

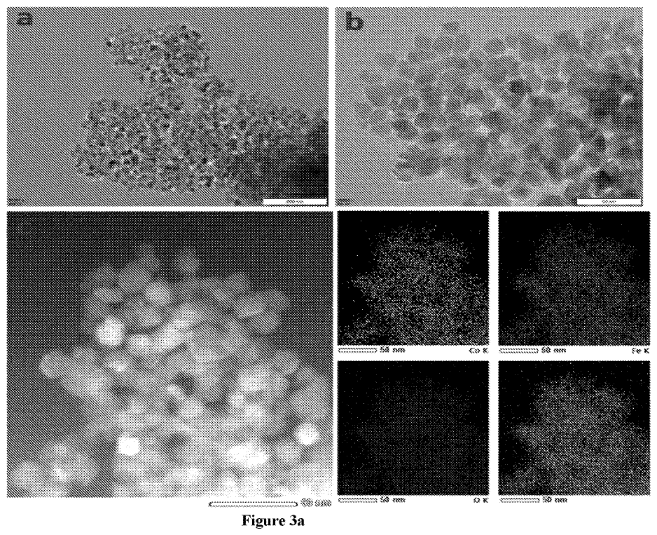

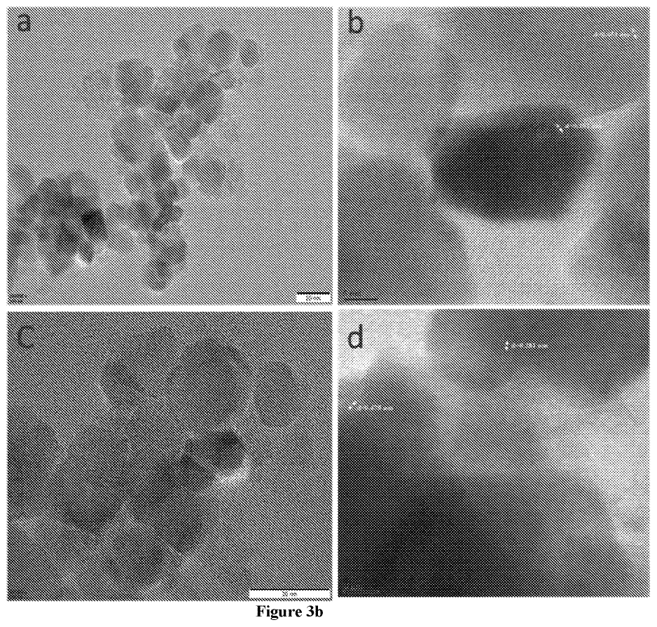

HRTEM images (FIG. 3a and FIG. 3b) of CoFe₂O₄ exhibit arrays of highly ordered and well crystallinity of cubic fd-3m symmetry, which are reliable with the XRD results. Moreover, HR-TEM images of these catalysts, revealing the high crystalline nature and uniform structure formation of CoFe₂O₄ catalyst. The HAADF STEM images prove that Co, Fe, and O are uniformly present and overlay mapping indicates that iron dispersion is more because of the high loading of iron in ferrite.

HR-TEM images of fresh and spent catalyst analysis are shown in FIG. 3b, and no changes are seen in the structure before and after the reaction. The HRTEM images exhibited that the lattice fringes of {311} and {111} set of planes with a d-spacing of 0.28 nm and 0.45 nm, respectively. It shows clear evidence for the formation of pure ferrite structure without any other phases.

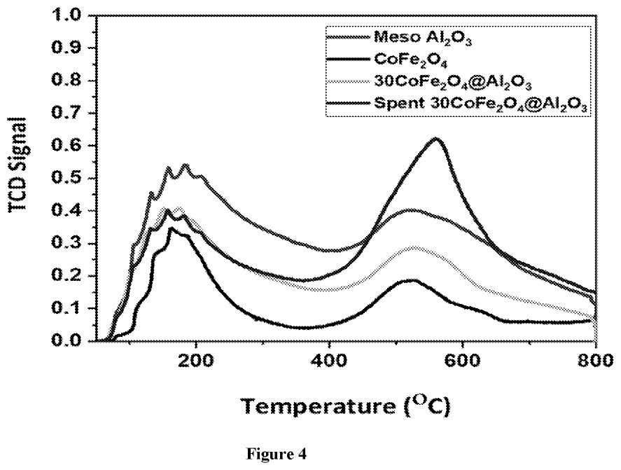

To identify the acidic sites, NH₃ temperature programmed desorption study as shown in FIG. 4 is carried out. All the catalysts containing both weak and strong acidic site corresponds to the peaks around 200° C. and 500° C. respectively. The mesoporous support is showing higher amount of weak and strong acidic sites around 160° C. and 520° C., respectively in comparison to ferrites supported on mesoporous Al₂O₃. It indicates that CoFe₂O₄ is highly dispersed on support. As per literature report, Bronsted and Lewis acidic sites and redox properties of catalysts are playing an important role in the activation of the C—H bond. In the present invention, the activation of methane happens around 200° C., which is clear evidence that the activation of methane occurs at weak acidic sites, and it is well-matched with the NH₃ TPD.

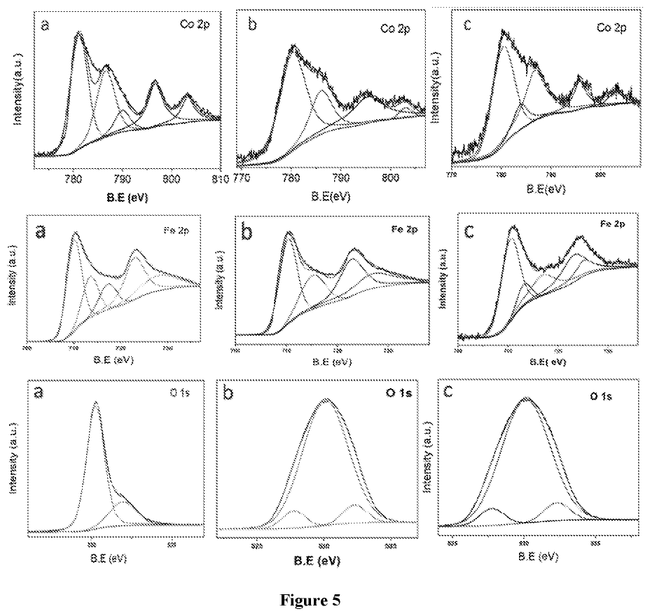

XPS study has been performed to identify the surface modification in terms of change in the oxidation of catalysts system. The difference in the oxidation state is due to the different coordination of metal-oxygen bonds. In general, A is in the +2-oxidation state located in tetrahedral coordination, and B is in the +3-oxidation state located in the octahedral coordination sites. In the inverse spinel structure, A is fully in octahedral coordination sites and B occupies both tetrahedral and octahedral sites. It is observed that, in all the ferrite catalysts of Co 2p core level spectra are shown in FIG. 5a. The peaks at 780.4 and 782.8 eV correspond to the tetrahedral coordinated of ferrite structure. In Fe 2p spectra shown in FIG. 5b the 2p 3/2 peaks at 710.3 and 713.6 eV corresponds to Fe³⁺ octahedral and tetrahedral sites. In all the catalysts, octahedral Fe³⁺ oxidation states are more in comparison with the tetrahedral site. In case of CoFe₂O₄ supported on mesoporous alumina catalysts there is a decrease in the tetrahedral Fe³⁺ due to the isomorphic substitution of Fe³⁺ ion. FIG. 5c is O 1s spectra exhibiting that the CoFe₂O₄ catalyst have binding energy at 530.1 and 532.3 eV corresponds to lattices oxygen and surface hydroxyl species. It is observed that the lower binding energy at 527.6 eV which corresponds to the formation of nucleophilic oxygen bounded to ferrite structure in the CoFe₂O₄ on a mesoporous alumina support.

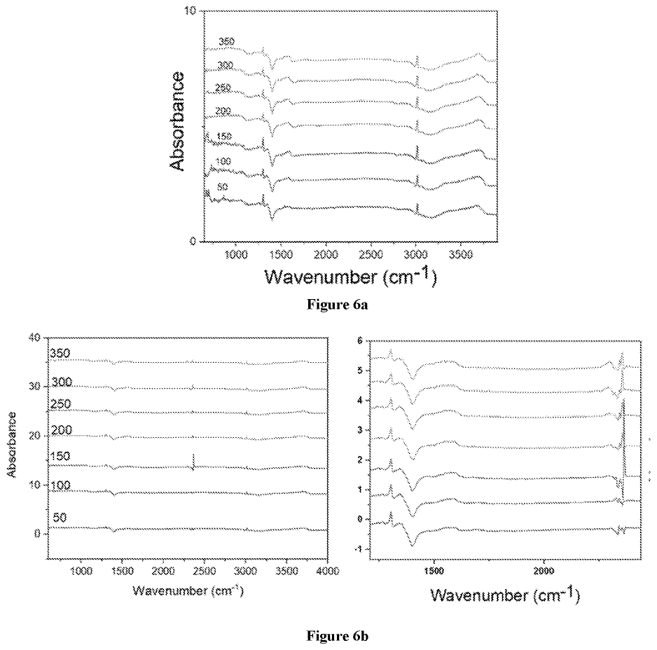

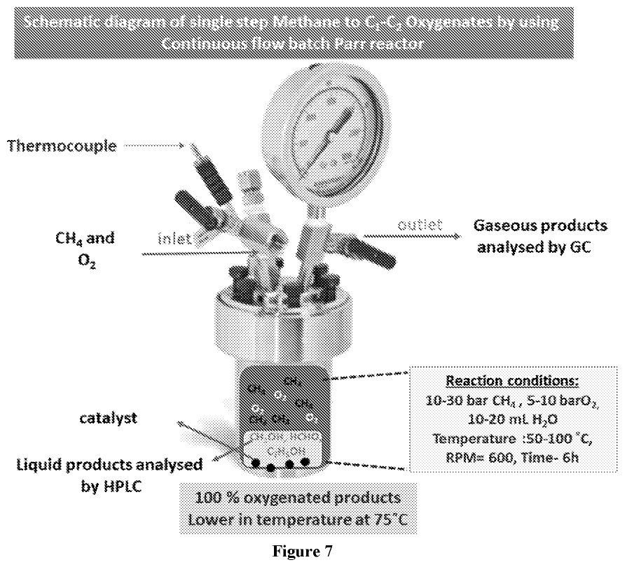

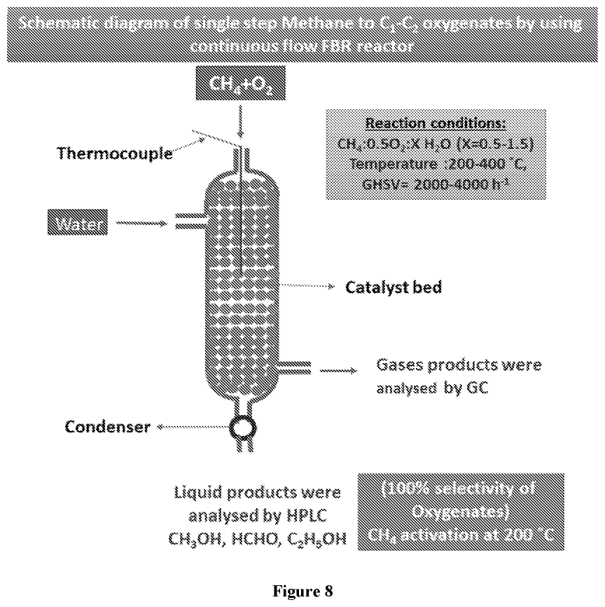

To identify the formation of intermediates of methane oxidation reaction, in-situ FT IR absorption spectra of as synthesized catalysts is done with CH₄ and O₂ at a different temperature ranging from 50-350° C. shown in FIG. 6a. In this case, it is observed that the formation of broad peaks around 1538-1578 cm⁻¹ indicating the formation HCO³⁻/C—C intermediates and the broad peak at the range of 2910-3000 cm⁻¹ indicating the formation of methoxy species. The in-situ FT IR spectrum shows that the methane is absorbed at 150° C., indicating that the activation of methane happens at lower temperature at 150° C.

In-situ FTIR absorbance spectra of CH₄ oxidation by using CO₂ as oxidation reactions are shown in the FIG. 6b. In this figure it is observed that the formation of new peak at 2291 cm⁻¹ corresponds to the formation of carbonates species and the intensity of CO₂ peak decreased with an increase in the temperature. The formation of methoxy species is observed at 2821 cm⁻¹ and a broad peak around 1530 cm⁻¹ after 200° C. of the reaction. The peak at 3711 cm⁻¹ is also increasing with an increase in the temperature, which indicates the formation of water as a side product in reaction conditions.

Several reactions are conducted by using O₂ as an oxidant in continuous mode reaction for CoFe₂O₄ catalyst and CoFe₂O₄ on Al₂O₃ catalyst. Results are summarised below in Table 2 and Table 3, respectively.

TABLE 2
Continuous mode reaction with O₂ as oxidant for CoFe₂O₄ catalyst

| | | | Reaction conditions: | | |
|---|---|---|---|---|---|
| | | | CH₄:O₂:H₂O = 1:0.5:1, GHSV = 3000 h⁻¹ | | |
| | | | Catalyst: | | |
| | | Conversion | CoFe₂O₄ | | |
| S. | Temp | (CH₄) | MeOH | HCHO | EtOH |
| No | ° C. | % | Mmol | mmol | mmol |
| 1 | 200 | 1.21 | 0.5247 | 0.21 | 0 |
| 2 | 250 | 8.98 | 2.1554 | 0.44 | 0 |
| 3 | 300 | 8.52 | 3.0335 | 0.620 | 0 |
| 4 | 350 | 10.33 | 2.6924 | 0.371 | 0 |
| 5 | 400 | 10.55 | 2.476 | 0.124 | 0 |
| 6 | 450 | 10.86 | 1.999 | 0.19048 | 8.6 |

TABLE 3
Continuous mode reaction with O₂ as
oxidant for CoFe₂O₄ on Al₂O₃ catalyst

| | | | Reaction conditions: | | |
|---|---|---|---|---|---|
| | | | CH₄:O₂:H₂O = 1:0.5:1, GHSV = 3000 h⁻¹ | | |
| | | | Catalyst: | | |
| | | Conversion | CoFe₂O₄ on Al₂O₃ | | |
| S. | Temp | (CH4) | MeOH | HCHO | EtOH |
| No | ° C. | % | Mmol | mmol | mmol |
| 1 | 200 | 8.88 | 3.2145 | 0.333 | 0 |
| 2 | 250 | 9.62 | 3.0653 | 0.6871 | 0 |
| 3 | 300 | 8.47 | 2.946 | 0.8197 | 0 |
| 4 | 350 | 10.26 | 2.834 | 0.9694 | 0 |
| 5 | 400 | 13.98 | 2.9385 | 1.1735 | 8.2 |
| 6 | 450 | 11.05 | 2.991 | 0.6259 | 5.3 |

Table 2 and 3 represent the continuous mode of reactions with O₂ as an oxidant for both the catalysts. The conversion of methane over CoFe₂O₄ catalyst observed ranges from 8.9 to 10.9%, and it has produced around 3 mmoles of methanol per gram of catalyst. In case of a higher temperature of 450° C. it produces around 8.63 mmoles of ethanol per gram of catalyst along with 1.9 mmoles of methanol and 0.1905 mmoles of formaldehyde.

This phenomenon clearly explains that at higher temperatures, the reaction is favourable for the C—C coupling to give ethanol. However, the catalyst with support showing a higher conversion of 8.88 to 13.980. The formation of formaldehyde is directly proportional to temperature, indicates higher temperature leads to the over oxidation products.

Tables 4 and 5 represent the continuous mode reactions with CO₂ as an oxidant for both the catalysts. The conversion of methane and CO₂ ranges from 4.1 to 10.2% and around 600, respectively, in the case of CoFe₂O₄. It is observed that a certain amount of oxygen is being produced during the course of the reaction, which has been detected in GC. These products explain to us that the CO₂ has been utilized or decomposed into oxygen and other carbonyl group species in the presence of water, which is being reflected in the higher conversion rates of CO₂. In case of the CoFe₂O₄ on Al₂O₃ catalysts shows the conversion from 3.5 to 11.8%, but we could observe that the formation of formaldehyde at higher temperature indicates over oxidation of methanol to form HCHO.

TABLE 4
Continuous mode reaction with
CO₂ as an oxidant for CoFe₂O₄ catalyst

| | | | Reaction conditions: | | | |
|---|---|---|---|---|---|---|
| | | | CH4:CO2:H2O = 1:1:1; GHSV = 3000 h⁻¹ | | | |
| | | | Catalyst: | | | |
| | | | CoFe₂O₄ | | | |
| | | Conversion | Conversion | | | |
| S. | Temp | (CH₄) | (CO₂) | MeOH | HCHO | EtOH |
| No | ° C. | % | % | mmol | mmol | mmol |
| 1 | 200 | 2.1 | 25 | 0.349 | 0.006 | 0 |
| 2 | 250 | 10.2 | 58 | 1.499 | 0.187 | 0 |
| 3 | 300 | 6.6 | 60.4 | 1.648 | 0.153 | 0 |
| 4 | 350 | 5.4 | 66.2 | 1.864 | 0.126 | 0 |
| 5 | 400 | 4.55 | 68.5 | 1.253 | 0.175 | 0 |
| 6 | 450 | 9.6 | 50.4 | 1.424 | 0.143 | 0 |

TABLE 5
Continuous mode reaction with CO₂ as
oxidant for CoFe₂O₄ on Al₂O₃ catalyst

| | | | Reaction conditions: | | | |
|---|---|---|---|---|---|---|
| | | | CH₄:CO₂:H₂O = 1:1:1; GHSV = 3000 h⁻¹ | | | |
| | | | Catalyst: | | | |
| | | | CoFe₂O₄ | | | |
| | | | on Al₂O₃ | | | |
| | | Conversion | Conversion | | | |
| S. | Temp | (CH₄) | (CO₂) | MeOH | HCHO | EtOH |
| No | ° C. | % | % | mmol | mmol | mmol |
| 1 | 200 | 3.5 | 50.68 | 0 | 0 | 0 |
| 2 | 250 | 5.6 | 51.18 | 1.909 | 4.2755 | 0 |
| 3 | 300 | 9.5 | 50.62 | 1.387 | 0.7851 | 0 |
| 4 | 350 | 8.6 | 53.87 | 1.036 | 1.2347 | 0 |
| 5 | 400 | 10.3 | 54.48 | 3.08 | 0.468 | 0 |
| 6 | 450 | 11.8 | 54.95 | 3.132 | 1.7415 | 0 |

Table 6 represents the batch mode reactions with O₂/CO₂/H₂O₂ as oxidants for CoFe₂O₄ on Al₂O₃ and CoFe₂O₄ catalysts. The reaction temperature was maintained at 75° C. for both O₂ and H₂O₂ and 150° C. for CO₂ as an oxidant. We observed a good selectivity of 22.1 mmoles of methanol per gram of catalyst, 11 mmoles per gram of catalyst towards ethanol and 4.8 mmoles per gram of catalyst towards formaldehyde with O₂ as oxidant over the catalyst with the support. It clearly states that the C—C coupling is more favourable in batch mode reactions along with formaldehyde. However, it explains that the catalyst is stable and it is capable of converting methane to value-added chemicals like methanol, ethanol and formaldehyde in presence of oxygen, carbon dioxide and hydrogen peroxide as soft oxidants.

TABLE 6
Batch mode reaction with O₂/CO₂/H₂O₂ as oxidants
over CoFe₂O₄ on Al₂O₃ and CoFe₂O₄ catalysts

| S. | | | Temp | Conv | Methanol | HCHO | Ethanol |
|---|---|---|---|---|---|---|---|
| No | Catalyst | Oxidants | ° C. | % | mmol | mmol | mmol |
| 1 | Mesoporous Al₂O₃ | O₂ | 75 | — | — | — | — |
| 2 | CoFe₂O₄ on Al₂O₃ | CO₂ | 75 | — | — | — | — |
| 3 | CoFe₂O₄ on Al₂O₃ | O₂ | 75 | 8.4 | 22.1 | 4.8 | 11 |
| 4 | CoFe₂O₄ on Al₂O₃ | CO₂ | 150 | 4.1 | 2.55 | 5.2 | 2.23 |
| 5 | CoFe₂O₄ on Al₂O₃ | H₂O₂ | 75 | 9.3 | 5.8 | 1.35 | 14.5 |
| 6 | CoFe₂O₄ | O₂ | 75 | 8.6 | 6.1 | 2.5 | 2 |
| 7 | CoFe₂O₄ | CO₂ | 150 | 5.2 | 1.3 | 2.3 | 1.4 |
| 8 | CoFe₂O₄ | H₂O₂ | 75 | 9.1 | 2.8 | 5.9 | 3.6 |

All the experimental results clearly showing that there are no over-oxidation products like CO₂, CO etc. In all the reactions, it is observed that small traces of ethane and ethene formation of C—C coupling due to CO₂ hydrogenation is taking place in the reaction over 275° C. The main advantage of this catalysts system is to activate both greenhouse gases (CH₄ and CO₂) to value-added products at a lower temperature.

### EXAMPLES

Following examples are given by way of illustration and therefore should not be construed to limit the scope of the invention.

### Example 1: Process for the Synthesis of Catalyst CoFe₂O₄

The CoFe₂O₄ inverse spinel catalysts were prepared by simple precipitation method by using NaOH as a precipitating agent. The molar ratios of (1:1, 1:2, and 2:1) metal precursors of iron and cobalt nitrates were dissolved in 50 ml of water and stirred for 2 h. The 6 M of 50 ml NaOH solution was added to the metal precursor solution dropwise. The black precipitate was formed while adding 6M NaOH solution indicates the formation of ferrite structure. The formed precipitate was aged for 2 h at 60° C. Then 3 M NaOH was added to get the pure form of CoFe₂O₄ without any impurities like Fe₂O₃ and Fe₃O₄. The final precipitate was filtered and dried in an oven at 100° C. for 24 h. The obtained powder was calcined at 550° C. in an air atmosphere for 6 h.

To prepare MnFe₂O₄, CuFe₂O₄, and CrFe₂O₄ metal oxide catalysts, in the process explained above, instead of iron precursor, manganese precursor, copper precursor and chromium precursor is used.

### Example 2: Process for the Synthesis of Mesoporous Alumina Support

The catalyst support was prepared by dissolving 2.0 g of P123 in 40 ml ethanol. Then, 6 g of PVP is added slowly into the dissolved solution under stirring. To this solution, 4.08 g of Aluminium Isopropoxide, 3.2 g 37% HCl and 0.9 g Citric acid were added under vigorous stirring. The resulting precipitate was aged for 12 h at 30° C. The final precipitate was calcined at 600° C. for 6 h (2° C./min).

Mesoporous zirconium oxide support is prepared by using the same procedure using Zirconium(IV) oxynitrate hydrate precursor.

Other supports like MCM-41, ZSM-5, and SBA-15 were synthesized from the previous literature known reports, e.g. 1. Prabu, Marimuthu, et al. ACS Omega 2019 4 (2), 3500-3507, DOI: 10.1021/acsomega.8b02547 (for SBA-15); 2. M. Liu et al. Chemical Engineering Journal 223 (2013) 678-687 (for MCM-41); and 3. Lingqian Meng et al., Chem. Mater. 2017, 29, 9, 4091-4096, DOI: 10.1021/acs.chemmater.7b00913 (ZSM-5).

### Example 3: Synthesis of the Catalyst with Support

The CoFe₂O₄ is supported on mesoporous alumina catalysts by physical mixing followed by the ball milling method. 30 wt % of CoFe₂O₄ was mixed with 1 g of mesoporous alumina support by using ball milling method for 1 h. After ball milling, the catalyst was calcined at 550° C. for 6 h. Similar procedure can be employed for synthesis of various metal oxides mentioned in example 1 on different supports mentioned in example 2.

### Example 4: General Procedure of Process for the Methane Oxidation Reaction

Methane oxidation reactions were carried out in both batch (Parr) and continuous flow (FBR) reactors. A high-temperature Inconel fixed bed reactor was employed for the continuous mode of the reaction. A 1:1:1 ratio of CH₄:CO₂:XH₂O and/or 1:0.5:0.5-1.5 ratio of CH₄:O₂:XH₂O (X=0.5-1.5) with 1 g of catalyst was used for the entire reaction with a temperature range from 200, 250, 300, 350, 400, 450 or 500° C. The gaseous products were analysed in Thermo Fisher Trace 1110 GC, and the liquid product has been analysed by Thermo Fisher HPLC (ISQ-EM) connected with H+ plus organic acid column with 0.005M H₂SO₄ as mobile phase.

A Parr setup was employed for the batch mode of the reaction. A 100 mg of the catalyst was immersed in 10 ml water then sealed properly. 10-30 bar methane and 5-10 bar oxygen filled in 50 ml reactor then heated at 75° C. or 100° C. for 6, 6.5, 7, 7.5, 8, 8.5, 9, 9.5 or 10 h. For CO₂ as an oxidant, 25 bar methane and 25 bar CO₂ were filled in a 50 ml reactor and heated at 100, 110, 125, 140 or 150° C. for 6 h. After 6 h, the reactor was cooled down to 4° C. then the liquid sample was collected and analyzed by HPLC, H+ plus column with 0.005 M H₂SO₄ as mobile phase, and the gaseous products were analyzed in Thermo Fisher Trace 1110 GC.

### ADVANTAGES OF THE INVENTION

Non-precious metals are incorporated in the catalyst system of the present invention.
Different oxidizing agents can be used along with the catalyst system of the present invention.
Inventors have demonstrated a single step conversion of methane into methanol and ethanol at low temperature and at atmospheric pressure.
Activation of alkanes at lower temperature is obtained.
Utilization of greenhouse gases can be achieved by applying the process of the present invention using this catalyst system.
The catalyst system is stable and scalable throughout the reactions.
Provides oxidation of alkane (e.g. methane) into oxygenated products (e.g. methanol, formaldehyde, ethanol) in single-step process at lower temperature (200° C.) and at atmospheric pressure, which is nowhere reported in the literature.
Novel methodology was used to prepare gamma alumina support as well as a new phase of CoxFexAl2-xO4 is formed in the catalyst.
No over-oxidation products were (COx) observed.
Selectivity of formation of oxygenated products is nearly 100%.

We claim:

1. A mixed metal oxide catalyst system for partial oxidation of (C₁-C₄) alkanes into value added products comprising:
i. spinel or inverse spinel structured mixed metal catalyst of formula AB₂O₄ supported onto a support;
wherein
A is a metal selected from the group consisting of manganese, cobalt, copper and chromium;
B is a metal of iron;
wherein the catalyst system exhibits array of ordered and crystallinity of cubic fd-3m symmetry.

2. The mixed metal oxide catalyst system as claimed in claim 1, wherein the catalyst is in a phase having formula of AxBxO₄ supported onto support, wherein the support is in phase of 2-x, and wherein x is in range of 0.01 to 0.5.

3. The mixed metal oxide catalyst system as claimed in claim 1, wherein the iron is in a ferrite form.

4. The mixed metal oxide catalyst system as claimed in claim 1, wherein the support is selected from the group consisting of mesoporous alumina, MCM-41, ZSM-5, SBA-15 and mesoporous zirconium oxide.

5. The mixed metal oxide catalyst system as claimed in claim 4, wherein the (C1-C4) alkane is selected from the group consisting of methane, ethane, propane and butane.

6. The mixed metal oxide catalyst system as claimed in claim 1, wherein the value-added product is selected from (C1-C6) alcohol and aldehyde, either alone or mixture thereof.

7. The mixed metal oxide catalyst system as claimed in claim 1, wherein the (C1-C6) alcohol is selected from the group consisting of methanol, ethanol, propanol, butanol, pentanol and hexanol or mixtures thereof.

8. The mixed metal oxide catalyst system as claimed in claim 1, wherein the aldehyde is selected from the group consisting of formaldehyde, acetaldehyde, propanal, butanal, and pentanal or mixture thereof.

9. The mixed metal oxide catalyst system as claimed in claim 1, wherein said catalyst system exhibit BET surface area in the range of 102.2 to 104.1 m²/g; average pore size in the range of 3.2 to 3.3 nm.

10. The mixed metal oxide catalyst system as claimed in claim 1, wherein AB₂O₄ is dispersed onto surface of said support; Metal A has octahedral coordination sites, and metal B has tetrahedral and octahedral sites in said catalyst system; and/or Metal A is in +2 oxidation state, and metal B is in +3 oxidation state.

11. A process for the preparation of mixed metal oxide catalyst system as claimed in claim 1, comprising the steps of:
a) dissolving the metal precursors in water in 1:1, 1:2, or 2:1 molar ratio under stirring for a period in the range of 1.5 to 3 hours to obtain a solution;
b) adding 4 to 7M NaOH solution into the solution as obtained in step a) to obtain a reaction mixture and aging the reaction mixture for 1.5 to 3 hours at a temperature in the range of 50 to 70° C.;
c) adding 2.5 to 4M NaOH solution into the reaction mixture obtained in step b) to obtain a product, followed by filtering and drying the product at a temperature in the range of 90 to 110° C. for a time period in the range of 20 to 26 hours and calcining at a temperature in the range of 520 to 580° C. in an air atmosphere for a time period in the range of 5 to 7 hours to obtained a catalyst without support;
d) mixing the catalyst obtained at step c) with support by using ball milling method for a time period in the range of 0.45 to 2 hours to obtain a catalyst with support; and
e) calcining the catalyst with support obtained at step da) at a temperature in the range of 500 to 600° C. for a time period in the range of 5 to 7 hours to get the mixed metal oxide catalyst system.

12. The process as claimed in claim 11, wherein the metal precursors at step a) is selected from the group consisting of iron nitrate, cobalt nitrate, Mn(NO₃)₂·4H₂O, Cu(NO₃)₂·3H₂O, and Cr (NO₃)₃·9H₂O.

13. A process for the partial oxidation of (C1-C4) alkane into value added products using the mixed metal oxide catalyst system as claimed in claim 1, the process comprising the steps of:
a) reacting the (C1-C4) alkane with an oxidant in a presence of water as solvent in a ratio ranging between 1:0.5:0.5 to 1:1:1.5 at a pressure of oxidant in range of 10-25 bar to obtain the value added products;
wherein the oxidant is selected from the group consisting of CO₂, O₂ and H₂O₂.

14. The process as claimed in claim 13, wherein the process is carried out in a batch mode in a Parr reactor(s) at a temperature in the range of 75-150° C. for a time period in the range of 5-7 hours or continuous mode in a continuous flow (FBR) reactor(s) at a temperature in the range of 150-500° C. for a period in the range of 12-24 hours.

15. The process as claimed in claim 13, wherein the pressure of (C1-C4) alkane is in the range of 25-30 bar; and a gas hourly space velocity is in the range of 2000 to 4000 h⁻¹.
